(19) United States
(12) Patent Application Publication
(10) Pub. No.: US 2009/0104240 A1
(43) Pub. Date: Apr. 23, 2009

(54) Dual Drug Formulations For Implantable Medical Devices For Treatment of Vascular Diseases

(75) Inventors: Gordon Stewart, San Francisco, CA (US); Syed F.A. Hossainy, Fremont, CA (US); Benjamyn Serna, Gilroy, CA (US); Mikael Trollsas, San Jose, CA (US); Jessica R. Desnoyer, San Jose, CA (US); Leslie Coleman, Redwood City, CA (US); Gina Zhang, Temecula, CA (US); Maricela Kezis, Sandy, UT (US)

Correspondence Address: SQUIRE, SANDERS & DEMPSEY LLP, 1 MARITIME PLAZA, SUITE 300, SAN FRANCISCO, CA, 94111, US

(73) Assignee: Abbott Cardiovascular Systems Inc., Santa Clara, CA (US)

(21) Appl. No.: 11/875,723

(22) Filed: Oct. 19, 2007

(51) Int. Cl. A61F 2/06; A61K 31/56; A61P 9/00

(52) U.S. Cl. 424/423; 623/1.42; 514/171

(57) ABSTRACT

The present invention relates to implantable medical devices coated with the polymer containing at least an olimus and a corticosteroid for the treatment of vascular diseases.

### FIELD OF THE INVENTION

This invention relates to the fields of organic chemistry, pharmaceutical chemistry, polymer science, material science and medicine. In particular, it relates to a medical device and method using a dual drug formulation for treating vascular diseases.

### BACKGROUND OF THE INVENTION

Until the mid-1980s, the accepted treatment for atherosclerosis, i.e., narrowing of the coronary artery(ies) was coronary by-pass surgery. While being quite effective and having evolved to a relatively high degree of safety for such an invasive procedure, by-pass surgery still involves potentially serious complications and in the best of cases an extended recovery period.

With the advent of percutaneous transluminal coronary angioplasty (PTCA) in 1977, the scene changed dramatically. Using catheter techniques originally developed for heart exploration, inflatable balloons were employed to re-open occluded regions in arteries. The procedure was relatively non-invasive, took a very short time compared to by-pass surgery and the recovery time was minimal. However, PTCA brought with it other problems such as vasospasm and elastic recoil of the stretched arterial wall which could undo much of what was accomplished and, in addition, it created a new problem, restenosis, the re-clogging of the treated artery due to neointimal hyperplasia.

The next improvement, advanced in the mid-1980s, was the use of a stent to maintain the luminal diameter after PTCA. This for all intents and purposes put an end to vasospasm and elastic recoil but did not entirely resolve the issue of restenosis. That is, prior to the introduction of stents, restenosis occurred in from about 30 to 50% of patients undergoing PTCA. Stenting reduced this to about 15 to 20%, much improved but still more than desirable.

In 2003, drug-eluting stents or DESs were introduced. The drugs initially employed with the DES were cytostatic compounds, that is, compounds that curtailed the proliferation of cells that resulted in restenosis. The occurrence of restenosis was thereby reduced to about 5 to 7%, a relatively acceptable figure. However, the use of DESs engendered yet another complication, late stent thrombosis, the forming of blood clots long after the stent was in place. It was hypothesized that the formation of blood clots was most likely due to delayed healing, a side-effect of the use of cytostatic drugs.

It has been found that the physiopathology of restenosis involves early injury to smooth muscle cells (SMCs), de-endothelialization and thrombus deposition. Over time, this leads to SMC proliferation and migration and extra-cellular matrix deposition. There is an increasing body of evidence suggesting that inflammation plays a pivotal role in linking this early vascular injury with neointimal growth and eventual lumen compromise, i.e., restenosis. Further, it has been observed that, when stenting is used, the inflammatory state if often more intense and prolonged thus exacerbating the preceding effects.

What is needed is an implantable medical device and method that deals with both the latter stage neointimal growth and the more immediate inflammatory process. The current invention provides such a device and method.

### SUMMARY OF THE INVENTION

Thus, in one aspect, the current invention relates to an implantable medical device, comprising:

a device body having an exposed surface; and,
a drug reservoir layer disposed over the exposed surface of the device body, the drug reservoir layer comprising a biocompatible polymer and at least two therapeutic agents, wherein the first therapeutic agent is an olimus and the second therapeutic agent is corticosteroid, the olimus to polymer wt/wt ratio is from about 1:30 to about 1:1, the corticosteroid to polymer wt/wt ratio is from about 1:30 to about 1:1 and the olimus and corticosteroid are substantially homogeneously dispersed in the polymer layer at an olimus to corticosteroid wt/wt ratio of from about 1:0.1 to about 1:0.5.

In an aspect of this invention, the corticosteroid is low potency.

In an aspect of this invention, the corticosteroid is medium to high potency.

In an aspect of this invention, the olimus is everolimus.

In an aspect of this invention, the corticosteroid is dexamethasone.

In an aspect of this invention, the medium to high potency corticosteroid is mometasone furoate.

In an aspect of this invention, the device body is a stent.

In an aspect of this invention, the biocompatible polymer is selected from the group consisting of poly(L-lactide), poly(D-lactide), poly(D,L-lactide), poly(meso-lactide), poly(L-lactide-co-glycolide), poly(D-lactide-co-glycolide), poly (D,L-lactide-co-glycolide), poly(meso-lactide-co-glycolide), poly(caprolactone), poly(hydroxyvalerate), poly(hydroxybutyrate), poly(ethylene glycol-co-butylene terephthalate), a fluoropolymer, a silicon polymer, aliphatic polyester, poly(acrylate) and poly(methacrylate).

In an aspect of this invention, the biocompatible polymer is poly(vinylidene fluoride-co-hexafluoropropylene).

In an aspect of this invention, the olimus drug loading is from about 10 microgram/cm² to about 300 microgram/cm².

An aspect of this invention is a method of treating a vascular disease using the above described implantable medical device.

In an aspect of this invention, in the above method, the olimus is everolimus.

In an aspect of this invention, in the above method, the corticosteroid is dexamethasone.

In an aspect of this invention, in the above method, the corticosteroid is mometasone furoate.

In an aspect of this invention, in the above method, the implantable medical device is a stent.

In an aspect of this invention, in the above method, olimus loading is from about 10 microgram/cm² to about 300 microgram/cm².

In an aspect of this invention, in the above method, the biocompatible polymer is selected from the group consisting of poly(L-lactide), poly(D-lactide), poly(D,L-lactide), poly(meso-lactide), poly(L-lactide-co-glycolide), poly(D-lactide-co-glycolide), poly (D,L-lactide-co-glycolide), poly(meso-lactide-co-glycolide), poly(caprolactone), poly(hydroxyvalerate), poly(hydroxybutyrate), poly(ethylene glycol-co-butylene terephthalate), a fluoropolymer, a silicon polymer, an aliphatic polyester, a poly(acrylate) and a poly(methacrylate).

In an aspect of this invention, the vascular disease is selected from the group consisting of atherosclerosis, restenosis, vulnerable plaque, peripheral vascular disease and late stent thrombosis.

In an aspect of this invention, the poly(vinylidene fluoride-co-hexafluoropropylene) is semicrystalline.

In an aspect of this invention, the poly(vinylidene fluoride-co-hexafluoropropylene) constitutional unit wt/wt ratio is about 85:15.

In an aspect of this invention, the poly(vinylidene fluoride-co-hexafluoropropylene) polymer has a average molecular weight from about 50,000 to about 500,000 Daltons.

In an aspect of this invention, the drug reservoir layer has a coating thickness from about 1 um to about 20 um.

In an aspect of this invention, the implantable medical device further comprising a primer layer disposed between the exposed surface of the device body and the reservoir layer.

In an aspect of this invention, the primer layer comprises acrylate or methacrylate polymer.

In an aspect of this invention, the acrylate or methacrylate polymer comprises poly(butyl methacrylate).

### DETAILED DESCRIPTION OF THE INVENTION

Use of the singular herein includes the plural and visa versa unless expressly stated to be otherwise. That is, “a” and “the” refer to one or more of whatever the word modifies. For example, “a therapeutic agent” includes one such agent, two such agents or, under the right circumstances, an even greater number of therapeutic agents. Likewise, “the layer” may refer to one, two or more layers and “the polymer” may mean one polymer or a plurality of polymers. By the same token, words such as, without limitation, “layers” and “polymers” refer to one layer of polymer as well as to a plurality of layers or polymers unless, again, it is expressly stated or obvious from the context that such is not intended.

As used herein, words of approximation such as, without limitation, “about” “substantially,” “essentially” and approximately mean that the feature so modified need not be exactly that which is expressly described but may vary from that written description to some extent. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±15%.

As used herein, “homogeneously dispersed” refers to the condition in which one or more substances are mixed together such that a sample taken from any location in the mixture will have the same chemical composition as a sample taken from any other location in the mixture.

As used herein, an “implantable medical device” refers to any type of appliance that is totally or partly introduced, surgically or medically, into a patient's body or by medical intervention into a natural orifice, and which is intended to remain there after the procedure. The duration of implantation may be essentially permanent, i.e., intended to remain in place for the remaining lifespan of the patient; until the device biodegrades; or until it is physically removed. Examples of implantable medical devices include, without limitation, implantable cardiac pacemakers and defibrillators; leads and electrodes for the preceding; implantable organ stimulators such as nerve, bladder, sphincter and diaphragm stimulators, cochlear implants; prostheses, vascular grafts, self-expandable stents, balloon-expandable stents, stent-grafts, grafts, artificial heart valves and cerebrospinal fluid shunts.

While implantable medical devices can serve several concurrent purposes and such stents are within the scope of this invention, an implantable medical device specifically designed and intended solely for the localized delivery of a therapeutic agent is likewise within the scope of this invention.

As used herein, “device body” refers to a fully formed implantable medical with an outer surface to which no coating or layer of material different from that of which the device itself is manufactured has been applied. A common example of a device body is a bare metal stent (BMS), which, as the name implies, is a fully-formed usable stent that has not been coated with a layer of any material different from the metal of which it is made on any surface that is in contact with bodily tissue or fluids. Of course, device body refers not only to BMSs but to any uncoated device regardless of what it is made of.

As used herein “exposed surface” refers to any surface of an implantable medical device of this invention however spatially oriented that is in contact with bodily tissue or fluids.

Implantable medical devices made of virtually any material, i.e., materials presently known to be useful for the manufacture of implantable medical devices and materials that may be found to be so in the future, may be used with a coating of this invention. For example, without limitation, an implantable medical device useful with this invention may be made of one or more biocompatible metals or alloys thereof including, but not limited to, cobalt-chromium alloy (ELGILOY, L-605), cobalt-nickel alloy (MP-35N), 316L stainless steel, high nitrogen stainless steel, e.g., BIODUR 108, nickel-titanium alloy (NITINOL), tantalum, platinum, platinum-iridium alloy, gold and combinations thereof.

Implantable medical devices may also be made of polymers that are biocompatible and biostable or biodegradable, the latter term including bioabsorbable and/or bioerodable.

As used herein, “biocompatible” refers to a polymer that both in its intact as synthesized state and in its decomposed state, i.e., its degradation products, is not, or at least is minimally toxic to living tissue; does not, or at least minimally and reparably injures living tissue; and/or does not, or at least minimally and/or controllably causes an immunological reaction in living tissue. Biocompatible polymers of this invention may be biostable or biodegradable where “biodegradable” simply means that the polymer will be decomposed over time when exposed to a physiological environs, i.e. to the conditions present in a patients body such as pH, the presence of enzymes, body temperature, etc.

Examples of biocompatible, relatively biostable polymers that may be used with an implantable medical device of this invention include, without limitation, polyacrylates, polymethacryates, polyureas, polyurethanes, polyolefins, polyvinylhalides, polyvinylidenehalides, polyvinylethers, polyvinylaromatics, polyvinylesters, polyacrylonitriles, polysiloxanes, alkyd resins and epoxy resins.

Biocompatible, biodegradable polymers include naturally-occurring polymers such as, without limitation, collagen, chitosan, alginate, fibrin, fibrinogen, cellulosics, starches, dextran, dextrin, hyaluronic acid, heparin, glycosaminoglycans, polysaccharides and elastin.

One or more synthetic or semi-synthetic biocompatible, biodegradable polymers may also be used to fabricate an implantable medical device useful with this invention. As used herein, a synthetic polymer refers to one that is created wholly in the laboratory while a semi-synthetic polymer refers to a naturally-occurring polymer that has been chemically modified in the laboratory. Examples of synthetic polymers include, without limitation, polyphosphazines, polyphosphoesters, polyphosphoester urethane, polyhydroxyacids, polyhydroxyalkanoates, polyanhydrides, polyesters, polyorthoesters, polyamino acids, polyoxymethylenes, poly(ester-amides) and polyimides.

Other biocompatible biodegradable polymers that may be used with the device and method of this invention include, without limitations, polyesters, polyhydroxyalkanoates (PHAs), poly(ester amides) that may optionally contain alkyl, amino acid, PEG and/or alcohol groups, polycaprolactone, poly(L-lactide), poly(D,L-lactide), poly(D,L-lactide-co-PEG) block copolymers, poly(D,L-lactide-co-trimethylene carbonate), polyglycolide, poly(lactide-co-glycolide), polydioxanone (PDS), polyorthoester, polyanhydride, poly(glycolic acid-co-trimethylene carbonate), polyphosphoester, polyphosphoester urethane, poly(amino acids), polycyanoacrylates, poly(trimethylene carbonate), poly(iminocarbonate), polycarbonates, polyurethanes, copoly(ether-esters) (e.g. PEO/PLA), polyalkylene oxalates, polyphosphazenes, PHA-PEG, and combinations thereof. The PHA may include poly(α-hydroxyacids), poly(β-hydroxyacid) such as poly(3-hydroxybutyrate) (PHB), poly(3-hydroxybutyrate-co-valerate) (PHBV), poly(3-hydroxyproprionate) (PHP), poly(3-hydroxyhexanoate) (PHH), or poly(4-hydroxyacid) such as poly poly(4-hydroxybutyrate), poly(4-hydroxyvalerate), poly(4-hydroxyhexanoate), poly(hydroxyvalerate), poly(tyrosine carbonates), poly(tyrosine arylates), poly(ester amide), polyhydroxyalkanoates (PHA), poly(3-hydroxyalkanoates) such as poly(3-hydroxypropanoate), poly(3-hydroxybutyrate), poly(3-hydroxyvalerate), poly(3-hydroxyhexanoate), poly(3-hydroxyheptanoate) and poly(3-hydroxyoctanoate), poly(4-hydroxyalkanaote) such as poly(4-hydroxybutyrate), poly(4-hydroxyvalerate), poly(4-hydroxyhexanote), poly(4-hydroxyheptanoate), poly(4-hydroxyoctanoate) and copolymers including any of the 3-hydroxyalkanoate or 4-hydroxyalkanoate monomers described herein or blends thereof, poly(D,L-lactide), poly(L-lactide), polyglycolide, poly(D,L-lactide-co-glycolide), poly(L-lactide-co-glycolide), polycaprolactone, poly(lactide-co-caprolactone), poly(glycolide-co-caprolactone), poly(dioxanone), poly(ortho esters), poly(anhydrides), poly(tyrosine carbonates) and derivatives thereof, poly(tyrosine ester) and derivatives thereof, poly(imino carbonates), poly(glycolic acid-co-trimethylene carbonate), polyphosphoester, polyphosphoester urethane, poly(amino acids), polycyanoacrylates, poly(trimethylene carbonate), poly(iminocarbonate), polyphosphazenes, silicones, polyesters, polyolefins, polyisobutylene and ethylene-alphaolefin copolymers, acrylic polymers and copolymers, vinyl halide polymers and copolymers, such as polyvinyl chloride, polyvinyl ethers, such as polyvinyl methyl ether, polyvinylidene halides, such as polyvinylidene chloride, polyacrylonitrile, polyvinyl ketones, polyvinyl aromatics, such as polystyrene, polyvinyl esters, such as polyvinyl acetate, copolymers of vinyl monomers with each other and olefins, such as ethylene-methyl methacrylate copolymers, acrylonitrile-styrene copolymers, ABS resins, and ethylene-vinyl acetate copolymers, polyamides, such as Nylon 66 and polycaprolactam, alkyd resins, polycarbonates, polyoxymethylenes, polyimides, polyethers, poly(glyceryl sebacate), poly(propylene fumarate), poly(n-butyl methacrylate), poly(sec-butyl methacrylate), poly(isobutyl methacrylate), poly(tert-butyl methacrylate), poly(n-propyl methacrylate), poly(isopropyl methacrylate), poly(ethyl methacrylate), poly(methyl methacrylate), epoxy resins, polyurethanes, rayon, rayontriacetate, cellulose acetate, cellulose butyrate, cellulose acetate butyrate, cellophane, cellulose nitrate, cellulose propionate, cellulose ethers, carboxymethyl cellulose, polyethers such as poly(ethylene glycol) (PEG), copoly(ether-esters) (e.g. poly(ethylene oxide-co-lactic acid) (PEO/PLA)), polyalkylene oxides such as poly(ethylene oxide), poly(propylene oxide), poly(ether ester), polyalkylene oxalates, phosphoryl choline containing polymer, choline, poly(aspirin), polymers and co-polymers of hydroxyl bearing monomers such as 2-hydroxyethyl methacrylate (HEMA), hydroxypropyl methacrylate (HPMA), hydroxypropylmethacrylamide, PEG acrylate (PEGA), PEG methacrylate, methacrylate polymers containing 2-methacryloyloxyethylphosphorylcholine (MPC) and n-vinyl pyrrolidone (VP), carboxylic acid bearing monomers such as methacrylic acid (MA), acrylic acid (AA), alkoxymethacrylate, alkoxyacrylate, and 3-trimethylsilylpropyl methacrylate (TMSPMA), poly(styreneisoprene-styrene)-PEG (SIS-PEG), polystyrene-PEG, polyisobutylene-PEG, polycaprolactone-PEG (PCL-PEG), PLA-PEG, poly(methyl methacrylate)-PEG (PMMA-PEG), polydimethylsiloxane-co-PEG (PDMS-PEG), poly(vinylidene fluoride)-PEG (PVDF-PEG), PLURONIC™ surfactants (polypropylene oxide-co-polyethylene glycol), poly(tetramethylene glycol), hydroxy functional poly(vinyl pyrrolidone), biomolecules such as collagen, chitosan, alginate, fibrin, fibrinogen, cellulose, starch, dextran, dextrin, hyaluronic acid, fragments and derivatives of hyaluronic acid, heparin, fragments and derivatives of heparin, glycosamino glycan (GAG), GAG derivatives, polysaccharide, elastin, elastin protein mimetics, or combinations thereof. Some examples of elastin protein mimetics include (LGGVG)ₙ, (VPGVG)ₙ, Val-Pro-Gly-Val-Gly, or synthetic biomimetic poly(L-glytanmate)-b-poly(2-acryloyloxyethyllactoside)-b-poly(l-glutamate) triblock copolymer.

In some embodiments of the current invention the polymer used with the device and in the method of this invention can be poly(ethylene-co-vinyl alcohol), poly(methoxyethyl methacrylate), poly(dihydroxylpropyl methacrylate), polymethacrylamide, aliphatic polyurethane, aromatic polyurethane, nitrocellulose, poly(ester amide benzyl), co-poly-{[N,N′-sebacoyl-bis-(L-leucine)-1,6-hexylene diester]_{0.75}-[N,N′-sebacoyl-L-lysine benzyl ester]_{0.25}} (PEA-Bz), co-poly-{[N,N′-sebacoyl-bis-(L-leucine)-1,6-hexylene diester]_{0.75}-[N,N′-sebacoyl-L-lysine-4-amino-TEMPO amide]_{0.25}} (PEA-TEMPO), aliphatic polyester, aromatic polyester, fluorinated polymers such as poly(vinylidene fluoride-co-hexafluoropropylene), poly(vinylidene fluoride) (PVDF), and Teflon™ (polytetrafluoroethylene), a biopolymer such as elastin mimetic protein polymer, star or hyper-branched SIBS (styrene-block-isobutylene-block-styrene), or combinations thereof. In some embodiments, where the polymer is a copolymer, it can be a block copolymer that can be, e.g., di-, tri-, tetra-, or oligo block copolymers or a random copolymer. In some embodiments, the polymer can also be branched polymers such as star polymers.

Presently preferred polymers for use with this invention include polyesters such as, without limitation, poly(L-lactide), poly(D-lactide), poly(D,L-lactide), poly(meso-lactide), poly(L-lactide-co-glycolide), poly(D-lactide-co-glycolide), poly (D,L-lactide-co-glycolide), poly(meso-lactide-co-glycolide), poly(caprolactone), poly(hydroxyvalerate), poly(hydroxybutyrate), poly(ethylene glycol-co-butylene terephthalate).

Other presently preferred polymers of this invention are fluoropolymers such as poly(vinylidene fluoride-co-hexafluoropropylene). When used, the poly(vinylidene fluoride-co-hexafluoropropylene) preferable at present has a constitutional unit weight-to-weight (wt/wt) ratio of about 85:15. The average molecular weight of the presently preferred poly(vinylidene fluoride-co-hexafluoropropylene) polymer is from about 50,000 to about 500,000 Daltons. Further, it is presently preferred that the poly(vinylidene fluoride-co-hexafluoropropylene) polymer used to form drug reservoir layer be semicrystalline. The presently preferred coating thickness of the poly(vinylidene fluoride-co-hexafluoropropylene) drug reservoir layer is from about 1 um to about 20 um.

As used herein, “constitutional unit” refers to a monomer-derived component of a polymer moiety. For example, a presently preferred polymer of the is invention is poly(vinylidene fluoride-co-hexafluoropropylene), which has the structure:

[(—CH₂CF₂—)ₘ/(—CF₂CF(CF₃)—)ₙ—]ₓ

is comprised of the constitutional units —CH₂CF₂—, derived from the monomer CH₂═CF₂, and —CF₂CF(CF₃)—, derived from the monomer CF₂═CFCF₃.

Blends and copolymers of the above polymers may also be used and are within the scope of this invention. Based on the disclosures herein, those skilled in the art will recognize those implantable medical devices and those materials from which they may be fabricated that will be useful with the coatings of this invention.

Presently preferred implantable medical devices of this invention are stents.

A stent refers generally to any device used to hold tissue in place in a patient's body. Particularly useful stents, however, are those used for the maintenance of the patency of a vessel in a patient's body when the vessel is narrowed or closed due to diseases or disorders including, without limitation, tumors (in, for example, bile ducts, the esophagus, the trachea/bronchi, etc.), benign pancreatic disease, coronary artery disease, carotid artery disease and peripheral arterial disease such as atherosclerosis, restenosis and vulnerable plaque. Vulnerable plaque (VP) refers to a fatty build-up in an arterial wall thought to be caused by inflammation. The VP is covered by a thin fibrous cap that can rupture leading to blood clot formation. A stent can be used to strengthen the wall of the vessel in the vicinity of the VP and act as a shield against such rupture. A stent can be used in, without limitation, neuro, carotid, coronary, pulmonary, aorta, renal, biliary, iliac, femoral and popliteal as well as other peripheral vasculatures. A stent can be used in the treatment or prevention of disorders such as, without limitation, thrombosis, restenosis, hemorrhage, vascular dissection or perforation, vascular aneurysm, chronic total occlusion, claudication, anastomotic proliferation, bile duct obstruction and ureter obstruction.

In addition to the above uses, stents may also be employed for the localized delivery of therapeutic agents to specific treatment sites in a patient's body. In fact, therapeutic agent delivery may be the sole purpose of the stent or the stent may be primarily intended for another use such as those discussed above with drug delivery providing an ancillary benefit.

A stent used for patency maintenance is usually delivered to the target site in a compressed state and then expanded to fit the vessel into which it has been inserted. Once at a target location, a stent may be self-expandable or balloon expandable. In any event, due to the expansion of the stent, any coating thereon must be flexible and capable of elongation.

As used herein, a “primer layer” refers to a coating consisting of a polymer or blend of polymers that exhibit good adhesion characteristics with regard to the material of which the device body is manufactured and good adhesion characteristic with regard to whatever material is to be coated on the device body. Thus, a primer layer serves as an intermediary layer between a device body and materials to be affixed to the device body and is, therefore, applied directly to the device body. Examples without limitation, of primers include acrylate and methacrylate polymers with poly(n-butyl methacrylate) being a presently preferred primer. Some additional examples of primers include, but are not limited to, poly(ethylene-co-vinyl alcohol), poly(vinyl acetate-co-vinyl alcohol), poly(methacrylates), poly(acrylates), polyethyleneamine, polyallylamine, chitosan, poly(ethylene-co-vinyl acetate), and parylene-C.

As use herein, a material that is described as a layer “disposed over” an indicated substrate be it a device body or another layer, refers to a coating of the material applied directly to the exposed surface of the indicated substrate. By “exposed surface” is meant any surface regardless of its physical location with respect to the configuration of the device that, in use, would be in contact with bodily tissues or fluids. “Disposed over” may, however, also refer to the application of the layer onto an intervening layer that has been applied to a stent body, wherein the layer is applied in such a manner that, were the intervening layer not present, the layer would cover substantially the entire exposed surface of the device body. An example of such an intervening layer is a primer layer.

As used herein, “drug reservoir layer” refers either to a layer of therapeutic agent applied neat or to a layer comprising a polymer that has dispersed within its threedimensional structure a therapeutic agent. A polymeric drug reservoir layer is designed such that, by one mechanism or another, e.g., without limitation, by elution or as the result of biodegradation of the polymer, the therapeutic substance is released from the layer into the surrounding environment. A drug reservoir layer may also act as rate-controlling layer.

As used herein, “rate-controlling layer” refers to a polymer layer that controls the release of therapeutic agent into the environment. As mentioned above, the drug reservoir layer may double as a rate-controlling layer. Alternatively, a separate rate-controlling layer comprising the same or a different polymer than that used in the drug reservoir layer may be disposed over the drug reservoir layer.

As used herein, “therapeutic agent” refers to any substance that, when administered in a therapeutically effective amount to a patient suffering from a disease, has a therapeutic beneficial effect on the health and well-being of the patient. A therapeutic beneficial effect on the health and well-being of a patient includes, but it not limited to: (1) curing the disease; (2) slowing the progress of the disease; (3) causing the disease to retrogress; or, (4) alleviating one or more symptoms of the disease. As used herein, a therapeutic agent also includes any substance that when administered to a patient, known or suspected of being particularly susceptible to a disease, in a prophylactically effective amount, has a prophylactic beneficial effect on the health and well-being of the patient. A prophylactic beneficial effect on the health and well-being of a patient includes, but is not limited to: (1) preventing or delaying on-set of the disease in the first place; (2) maintaining a disease at a retrogressed level once such level has been achieved by a therapeutically effective amount of a substance, which may be the same as or different from the substance used in a prophylactically effective amount; or, (3) preventing or delaying recurrence of the disease after a course of treatment with a therapeutically effective amount of a substance, which may be the same as or different from the substance used in a prophylactically effective amount, has concluded.

As used herein, “treating” refers to the administration of a therapeutically effective amount of a therapeutic agent to a patient known or suspected to be suffering from a vascular disease.

A “therapeutically effective amount” refers to that amount of a therapeutic agent that will have a beneficial affect, which may be curative or palliative, on the health and well-being of the patient with regard to the vascular disease with which the patient is known or suspected to be afflicted. A therapeutically effective amount may be administered as a single bolus, as intermittent bolus charges, as short, medium or long term sustained release formulations or as any combination of these. As used herein, short-term sustained release refers to the administration of a therapeutically effective amount of a therapeutic agent over a period from about several hours to about 3 days. Medium-term sustained release refers to administration of a therapeutically effective amount of a therapeutic agent over a period from about 3 day to about 14 days and long-term refers to the delivery of a therapeutically effective amount over any period in excess of about 14 days. Any reference a therapeutic agent relating to its presence on an implantable medical device or its use in a method of this invention is to be understood as referring to a therapeutically effective amount of that therapeutic agent.

The actual delivered dose necessary to achieve a therapeutically effective amount will be readily determinable by those of ordinary skill in the art based on the disclosures herein without undue experimentation. That is, the therapeutic agents set forth herein are primarily known FDA approved drugs, in particular for the purposes of this invention everolimus, dexamethasone and mometasome furoate, and their approved dosages are likewise known. It may be necessary to adjust the published dosages based on differences in the published delivery route and the localized delivery contemplated by this invention. For the purposes of this invention the dose will be described in terms of the therapeutic agent loading, that is the amount of the therapeutic agent that is dispersed in the drug reservoir layer. It is understood that the actual delivered dose is related to but is not exactly the same as the loading since not all of the therapeutic agent will necessarily be released from the drug reservoir layer and, further, the rate of release of the therapeutic agent coupled with the pharmacokinetics of the therapeutic agent will dictate how much therapeutic agent is actually present in a patient's tissues at any particular time. In any event, at present, loading dosages of the olimus and corticosteroid herein will generally be in the range of about 0.1 to about 1000 μg/cm², preferably about 0.5 to about 500 μg/cm² and presently most preferably from about 1 to about 300 μg/cm². Modification of these numbers based on actual clinical data is well within the capability of those of ordinary skill in the art without undue experimentation and any such modification is within the scope of this invention.

Presently preferred therapeutic agents of this invention are the olimus drugs and low or medium to high potency corticosteroids. Particularly preferred at present are medium to high potency corticosteroids.

As used herein, “olimus” refers to the family of drugs that included, without limitation, sirolimus (rapamycin), everolimus, zoltarolimus, Biolimus A9 (Biosensors International, Singapore), AP23572 (Ariad Pharmaceuticals), tacrolimus, pimecrolimus and derivates or analogs of any of these.

As used herein, a “corticosteroid” refers to a naturally occurring steroid hormone that is produced in the adrenal cortex and to synthetic therapeutic agents that exhibit corticosteroid-like pharmacological effects. Of particular importance to the methods of this invention is the ability of corticosteroids and their synthetic counterparts to mediate physiological systems related to the immune response and to regulation of inflammation.

As used herein, the relative potency of a corticosteroid is based on clinical studies and vasomotor assays. Corticosteroid potencies are generally placed in one of four classifications, very high (super high) potency, high potency, moderate or mid potency and low potency. The following is a non-exhaustive list of various corticosteroids by potency:

### Very High (Super High) Potency

augmented betamethasone dipropionate
diflorasone diacetate
halobetasol propionate
clobetasol propionate
flurandrenolide
halobetasol propionate

### High Potency

amcinonide
betamethasone dipropionate
desoximetasone
fluocinolone acetonide
halcinonide
triamcinolone acetonide

### Moderate (Mid) Potency

betamethasone benzoate
clocortolone pivalate
flurandrenolide
fluticasone propionate
hydrocortisone valerate
mometasone furoate
triamcinolone acetonide
prednicarbate
desonide

### Low Potency

aclometasone
dexamethasone
fluccinolone acetonide

For the purposes of this invention low, moderate and high potencies, in particular moderate to high potency corticosteroids, are presently preferred. While it has been found that super high potency clobetasone has a beneficial effect with regard to inflammation, impairment of vessel endothelialization was observed to be impaired.

In addition to an olimus and a corticosteroid, an implantable medical device of this invention and the method herein may comprise additional therapeutic agents that are known or become known as being effective in treating the various physiological events comprising vascular diseases.

For example, other therapeutic agents that may be useful with the implantable medical device and method of this invention include, without limitation, antiproliferative agents, anti-inflammatory agents, antineoplastics and/or antimitotics, antiplatelet, anticoagulant, antifibrin, and antithrombin drugs, cytostatic or antiproliferative agents, antibiotics, antiallergic agents and antioxidants.

Antiproliferative agents include, without limitation, actinomycin D, taxol, docetaxel, paclitaxel and perfenidone.

Anti-inflammatory agents include, without limitation, alclofenac, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clopirac, cortodoxone, deflazacort, diclofenac potassium, diclofenac sodium, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, morniflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicylic acid) and salicylic acid.

Anti-neoplastic and/or anti-mitotic agents include, without limitation, paclitaxel, docetaxel, methotrexate, azathioprine, vincristine, vinblastine, fluorouracil, doxorubicin hydrochloride, and mitomycin.

Antiplatelet, anticoagulant, antifibrin, and antithrombin agents include, without limitation, sodium heparin, low molecular weight heparins, heparinoids, hirudin, argatroban, forskolin, vapiprost, prostacyclin, prostacyclin dextran, D-phe-pro-arg-chloromethylketone, dipyridamole, glycoprotein IIb/IIIa platelet membrane receptor antagonist antibody, recombinant hirudin and thrombin, thrombin inhibitors such as Angiomax ä, calcium channel blockers such as nifedipine, colchicine, fish oil (omega 3-fatty acid), histamine antagonists, lovastatin, monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), nitric oxide or nitric oxide donors, super oxide dismutases, super oxide dismutase mimetic, 4-amino-2,2,6,6-tetramethylpiperidine-1-oxyl (4-amino-TEMPO) and estradiol.

Cytostatic or anti-proliferative agents include, without limitation, angiopeptin, angiotensin converting enzyme inhibitors such as captopril, cilazapril or lisinopril, calcium channel blockers such as nifedipine; colchicine, fibroblast growth factor (FGF) antagonists; fish oil (ω-3-fatty acid); histamine antagonists; lovastatin, monoclonal antibodies such as, without limitation, those specific for Platelet-Derived Growth Factor (PDGF) receptors; nitroprusside, phosphodiesterase inhibitors, prostaglandin inhibitors, suramin, serotonin blockers, steroids, thioprotease inhibitors, triazolopyrimidine (a PDGF antagonist) and nitric oxide.

Anti-allergenic agents include, without limitation, permirolast potassium.

Other potentially useful therapeutic agents include, without limitation, alpha-interferon, genetically engineered epithelial cells, DNA and RNA nucleic acid sequences, antisense molecules, and ribozymes, antibodies, receptor ligands, enzymes, adhesion peptides, blood clotting factors, inhibitors or clot dissolving agents such as streptokinase and tissue plasminogen activator, antigens for immunization, hormones and growth factors, oligonucleotides, retroviral vectors; antiviral agents; analgesics; anorexics; antihelmintics; antiarthritics, antiasthmatic agents; anticonvulsants; antidepressants; antidiuretic agents; antidiarrheals; antihistamines; antimigrain preparations; antinauseants; antiparkinsonism drugs; antipruritics; antipsychotics; antipyretics; antispasmodics; anticholinergics; sympathomimetics; xanthine derivatives; cardiovascular preparations including calcium channel blockers, beta-blockers such as pindolol, antiarrhythmics; antihypertensives; diuretics; vasodilators including general coronary; peripheral and cerebral; central nervous system stimulants; cough and cold preparations, including decongestants; hypnotics; immunosuppressives; muscle relaxants; parasympatholytics; psychostimulants; sedatives; tranquilizers; natural or genetically engineered lipoproteins; and restenosis reducing agents.

As used herein, a “vascular disease” refers to a disease of the vessels, primarily arteries and veins, which transport blood to and from the heart, brain and peripheral organs such as, without limitation, the arms, legs, kidneys and liver. In particular “vascular disease” refers to the coronary arterial system, the carotid arterial system and the peripheral arterial system. The disease that may be treated is any that is amenable to treatment with a therapeutic agent, either as the sole treatment protocol or as an adjunct to other procedures such as surgical intervention. The disease may be, without limitation, atherosclerosis, vulnerable plaque, restenosis or peripheral arterial disease.

“Atherosclerosis” refers to the depositing of fatty substances, cholesterol, cellular waste products, calcium and fibrin on the inner lining or intima of an artery. Smooth muscle cell proliferation and lipid accumulation accompany the deposition process. In addition, inflammatory substances that tend to migrate to atherosclerotic regions of an artery are thought to exacerbate the condition. The result of the accumulation of substances on the intima is the formation of fibrous (atheromatous) plaques that occlude the lumen of the artery, a process called stenosis. When the stenosis becomes severe enough, the blood supply to the organ supplied by the particular artery is depleted resulting is strokes, if the afflicted artery is a carotid artery, heart attack if the artery is a coronary artery, or loss of organ function if the artery is peripheral.

“Restenosis” refers to the re-narrowing or blockage of an artery at or near the site where angioplasty or another surgical procedure was previously performed to remove a stenosis. It is generally due to smooth muscle cell proliferation and, at times, is accompanied by thrombosis. Prior to the advent of implantable stents to maintain the patency of vessels opened by angioplasty, restenosis occurred in 40-50% of patients within 3 to 6 months of undergoing the procedure. Post-angioplasty restenosis before stents was due primarily to smooth muscle cell proliferation. There were also issues of acute reclosure due to vasospasm, dissection, and thrombosis at the site of the procedure. Stents eliminated acute closure from vasospasm and greatly reduced complications from dissections. While the use of IIb-IIIIa anti-platelet drugs such as abciximab and epifabatide, which are anti-thrombotic, reduced the occurrence of post-procedure clotting (although stent placement itself can initiate thrombosis). Stent placement sites are also susceptible to restenosis due to abnormal tissue growth at the site of implantation. This form of restenosis tends also to occur at 3 to 6 months after stent placement but it is not affected by the use of anti-clotting drugs. Thus, alternative therapies are continuously being sought to mitigate, preferably eliminate, this type of restenosis. Drug eluting stents (DES) which release a variety of therapeutic agents at the site of stent placement have been in use for some time. To date these stents comprised delivery interfaces (lengths) that are less than 40 mm in length and, in any event, have delivery interfaces that are not intended, and most often do not, contact the luminal surface of the vessel at the non-afflicted region at the periphery of the afflicted region.

“Vulnerable plaque” refers to an atheromatous plaque that has the potential of causing a thrombotic event and is usually characterized by a very thin wall separating it from the lumen of an artery. The thinness of the wall renders the plaque susceptible to rupture. When the plaque ruptures, the inner core of usually lipid-rich plaque is exposed to blood, with the potential of causing a potentially fatal thrombotic event through adhesion and activation of platelets and plasma proteins to components of the exposed plaque.

The phenomenon of vulnerable plaque has created new challenges in recent years for the treatment of heart disease. Unlike occlusive plaques that impede blood flow, vulnerable plaque develops within the arterial walls, but it often does so without the characteristic substantial narrowing of the arterial lumen which produces symptoms. As such, conventional methods for detecting heart disease, such as an angiogram, may not detect vulnerable plaque growth into the arterial wall.

The intrinsic histological features that may characterize a vulnerable plaque include increased lipid content, increased macrophage, foam cell and T lymphocyte content, and reduced collagen and smooth muscle cell (SMC) content. This fibroatheroma type of vulnerable plaque is often referred to as “soft,” having a large lipid pool of lipoproteins surrounded by a fibrous cap. The fibrous cap contains mostly collagen, whose reduced concentration combined with macrophage-derived enzyme degradation can cause the fibrous cap of these lesions to rupture under unpredictable circumstances. When ruptured, the lipid core contents, thought to include tissue factor, contact the arterial bloodstream, causing a blood clot to form that can completely block the artery resulting in an acute coronary syndrome (ACS) event. This type of atherosclerosis is coined “vulnerable” because of unpredictable tendency of the plaque to rupture. It is thought that hemodynamic and cardiac forces, which yield circumferential stress, shear stress, and flexion stress, may cause disruption of a fibroatheroma type of vulnerable plaque. These forces may rise as the result of simple movements, such as getting out of bed in the morning, in addition to in vivo forces related to blood flow and the beating of the heart. It is thought that plaque vulnerability in fibroatheroma types is determined primarily by factors which include: (1) size and consistency of the lipid core; (2) thickness of the fibrous cap covering the lipid core; and (3) inflammation and repair within the fibrous cap.

“Thrombosis” refers to the formation or presence of a blood clot (thrombus) inside a blood vessel or chamber of the heart. A blood clot that breaks off and travels to another part of the body is called an embolus. If a clot blocks a blood vessel that feeds the heart, it causes a heart attack. If a clot blocks a blood vessel that feeds to brain, it causes a stroke.

Peripheral vascular diseases are generally caused by structural changes in blood vessels caused by such conditions as inflammation and tissue damage. A subset of peripheral vascular disease is peripheral artery disease (PAD). PAD is a condition that is similar to carotid and coronary artery disease in that it is caused by the buildup of fatty deposits on the lining or intima of the artery walls. Just as blockage of the carotid artery restricts blood flow to the brain and blockage of the coronary artery restricts blood flow to the heart, blockage of the peripheral arteries can lead to restricted blood flow to the kidneys, stomach, arms, legs and feet.

While not being held to any particular theory, it is hypothesized that the dual therapeutic agent approach of this invention is particularly efficacious for the following reasons. Angioplasty and stent placement cause injury to the arteries which prompt the arteries to release inflammatory cytokines (chemical messages that tell the body it is under attack). This recruits the body's anti-inflammatory arsenal, including macrophages and other parts of the immune system which is followed by rapid growth in the number of smooth muscle. This is called neointimal hyperplasia which is part of what causes a blood vessel to reblock, which is termed as restenosis. Anti-proliferative therapeutic agents such as the olimus compounds, as exemplified by everolimus inhibit the proliferation of smooth muscle cells. The addition of an anti-inflammatory therapeutic agent such as a corticosteroid as exemplified by dexamethasone and mometasone furoate inhibits the production of inflammatory cytokines, thus suppressing the inflammatory response as well. Thus the dual drug-based formulations of this invention have shown to be advantageous over single drug-based formulations in preclinical models by demonstrating a reduction in the overall neointimal response, a reduction in the variability of the neointimal response, and a reduction in the overall inflammatory response and foreign body response.

The drug reservoir layer of the device and method of this invention comprises at least two therapeutic agents. The first therapeutic agent is an olimus, in particular at present everolimus, and the second therapeutic agent is a cortidosteroid, in particular at present dexamethasone or mometasone furoate.

The everolimus to polymer wt/wt ratio is from about 1:30 to about 1:1 and the dexamethasone or mometasone furoate to polymer wt/wt ratio is from about 1:30 to about 1:1. The everolimus and dexamethasone or memetasone furoate are dispersed in the polymer layer at a wt/wt ratio of from about 1:0.1 to about 1:0.5.

Table 1 shows the design of eight different everolimus/dexamethasone constructs. The “Dosage Description” shows the doses of everolimus and dexamethasone used (normalized for stent surface area). Actual amounts in micrograms (ug) are in the “Reservoir” column of the table. The eight arms are divided into four doses, with two drug:polymer (D:P) ratio for each dose.

TABLE 1
Everolimus/Dexamethasone DES Formulations

| | Coating Info | | | | | | | |
|---|---|---|---|---|---|---|---|---|
| | | EVR | DEX | Total | Reservoir | | | |
| | Dosage | Dose | Dose | Dose | | EVR | DEX | Reservoir |
| | Description | (ug/cm2) | (ug/cm2) | (ug/cm2) | D:P | Total(ug) | Total(ug) | Total(ug) |
| Arm 1 | 25:50 | 25 | 50 | 75 | 1:4.0 | 16 | 32 | 240 |
| Arm 2 | 25:50 | 25 | 50 | 75 | 1:8.0 | 16 | 32 | 432 |
| Arm 3 | 25:200 | 25 | 200 | 225 | 1:4.0 | 16 | 128 | 720 |
| Arm 4 | 25:200 | 25 | 200 | 225 | 1:6.0 | 16 | 128 | 1008 |
| Arm 5 | 100:50 | 100 | 50 | 150 | 1:3.0 | 64 | 32 | 384 |
| Arm 6 | 100:50 | 100 | 50 | 150 | 1:6.0 | 64 | 32 | 672 |
| Arm 7 | 100:200 | 100 | 200 | 300 | 1:3.0 | 64 | 128 | 768 |
| Arm 8 | 100:200 | 100 | 200 | 300 | 1:5.0 | 64 | 128 | 1152 |

EVR = Everolimus
DEX = Dexamethasone
D = Drug which can be either everolimus or dexamethasone
P = Polymer

Table 2 shows the recovery of both everolimus and dexamethasone from the stents. Recovery is calculated by dividing the amount of therapeutic agent recovered from a stent by the amount that was expected to be on the stent.

TABLE 2

| | Dose | | Everolimus | | | Dexamethasone | | |
|---|---|---|---|---|---|---|---|---|
| | (E:D) | D:P | Average | SD | RSD | Average | SD | RSD |
| Arm 1 | 25:50 | 1:4 | 80.2% | 3.4% | 4.2% | 90.9% | 0.7% | 0.8% |
| Arm 2 | 25:50 | 1:8 | 77.3% | 3.0% | 3.9% | 90.7% | 0.4% | 0.5% |
| Arm 3 | 25:200 | 1:4 | 63.1% | 3.1% | 4.8% | 88.8% | 0.3% | 0.3% |
| Arm 4 | 25:200 | 1:6 | 56.0% | 3.4% | 6.1% | 87.6% | 0.3% | 0.3% |
| Arm 5 | 100:50 | 1:3 | 89.3% | 1.5% | 1.7% | 86.7% | 0.4% | 0.5% |
| Arm 6 | 100:50 | 1:6 | 87.2% | 0.3% | 0.4% | 88.9% | 0.3% | 0.4% |
| Arm 7 | 100:200 | 1:3 | 89.1% | 0.9% | 1.0% | 94.0% | 0.2% | 0.3% |
| Arm 8 | 100:200 | 1:5 | 81.6% | 1.5% | 1.9% | 92.2% | 1.3% | 1.4% |

EVR = Everolimus
DEX = Dexamethasone
D = Drug which can be either everolimus or dexamethasone
P = Polymer
SD = standard deviation
RSD = relative standard deviation = SD/mean

Drug recovery was typically 85 to 100%, depending on several factors. All drug recoveries except for everolimus in Arm 3 and 4 have typical recovery. It is not clear why the recoveries were lower than expected, although it could be related to mixing or analysis.

While the present invention has been described in terms of certain embodiments, other embodiments not expressly disclosed will, based in the disclosure herein, occur to those skilled in the art. Such embodiments are within the scope of this invention.

What is claimed:

1. An implantable medical device, comprising:
a device body having an exposed surface;
a drug reservoir layer disposed over the exposed surface of the device body, the drug reservoir layer comprising a biocompatible polymer and at least two therapeutic agents, wherein the first therapeutic agent is an olimus and the second therapeutic agent is corticosteroid, the olimus to polymer wt/wt ratio is from about 1:30 to about 1:1, the corticosteroid to polymer wt/wt ratio is from about 1:30 to about 1:1 and the olimus and corticosteroid are substantially homogeneously dispersed in the polymer layer at an olimus to corticosteroid wt/wt ratio of from about 1:0.1 to about 1:0.5.

2. The implantable medical device of claim 1, wherein the corticosteroid is low potency.

3. The implantable medical device of claim 1, wherein the corticosteroid is medium to high potency.

4. The implantable medical device of claim 1, wherein the olimus is everolimus.

5. The implantable medical device of claim 2, wherein the corticosteroid is dexamethasone.

6. The implantable medical device of claim 3, wherein the medium to high potency corticosteroid is mometasone furoate.

7. The implantable medical device of claim 1, wherein the device body is a stent.

8. The implantable medical device of claim 1, wherein the biocompatible polymer is selected from the group consisting of poly(L-lactide), poly(D-lactide), poly(D,L-lactide), poly(meso-lactide), poly(L-lactide-co-glycolide), poly(D-lactide-co-glycolide), poly (D,L-lactide-co-glycolide), poly(meso-lactide-co-glycolide), poly(caprolactone), poly(hydroxyvalerate), poly(hydroxybutyrate), poly(ethylene glycol-co-butylene terephthalate), a fluoropolymer, a silicon polymer, aliphatic polyester, poly(acrylate) and poly(methacrylate).

9. The implantable medical device of claim 8, wherein the biocompatible polymer is poly(vinylidene fluoride-co-hexafluoropropylene).

10. The implantable medical device of claim 1, wherein the olimus drug loading is from about 10 microgram/cm² to about 300 microgram/cm².

11. A method of treating a vascular disease, comprising using the implantable medical device of claim 1.

12. The method of claim 11, wherein the olimus is everolimus.

13. The method of claim 12, wherein the corticosteroid is dexamethasone.

14. The method of claim 12, wherein the corticosteroid is mometasone furoate.

15. The method of claim 11, wherein the implantable medical device is a stent.

16. The method of claim 12, wherein olimus loading is from about 10 microgram/cm² to about 300 microgram/cm².

17. The method of claim 11, wherein the biocompatible polymer is selected from the group consisting of poly(L-lactide), poly(D-lactide), poly(D,L-lactide), poly(meso-lactide), poly(L-lactide-co-glycolide), poly(D-lactide-co-glycolide), poly (D,L-lactide-co-glycolide), poly(meso-lactide-co-glycolide), poly(caprolactone), poly(hydroxyvalerate), poly(hydroxybutyrate), poly(ethylene glycol-co-butylene terephthalate), a fluoropolymer, a silicon polymer, an aliphatic polyester, a poly(acrylate) and a poly(methacrylate).

18. The method of claim 11, wherein the vascular disease is selected from the group consisting of atherosclerosis, restenosis, vulnerable plaque, peripheral vascular disease and late stent thrombosis.
